(19) United States
(12) Patent Application Publication
(10) Pub. No.: US 2023/0116974 A1
(43) Pub. Date: Apr. 20, 2023

(54) Complexation of Curcumin with Calixarene Derivatives

(71) Applicant: University of Cincinnati, Cincinnati, OH (US)

(72) Inventors: Harshita Kumari, Loveland, OH (US); Xu Kang, Cincinnati, OH (US)

(73) Assignee: University of Cincinnati, Cincinnati, OH (US)

(21) Appl. No.: 17/850,931

(22) Filed: Jun. 27, 2022

Related U.S. Application Data
(60) Provisional application No. 63/215,120, filed on Jun. 25, 2021.

(51) Int. Cl. A61K 8/35; A61K 8/34; A61Q 19/02

(52) U.S. Cl. CPC A61K 8/35; A61K 8/347; A61Q 19/02; A61K 2800/805

(57) ABSTRACT

A complex of curcumin with a polyphenolic macrocyclic host is disclosed. The macrocyclic host is a resorcin[4]arene. In one approach, the polyphenolic macrocyclic host is calix[8]arene. In another approach, the polyphenolic macrocyclic host is tert-butylcalix[8]arene.

### CROSS REFERENCE TO RELATED APPLICATIONS

This application claims priority to U.S. Provisional Application Ser. No. 63/215,120, filed Jun. 25, 2021, which application is hereby incorporated by reference in its entirety.

### TECHNICAL FIELD

The present invention relates to complexes of curcumin.

### BACKGROUND OF THE INVENTION

Curcumin is the main active compound of turmeric and is widely used in the cosmetic industry, food industry, and as a supplement. Prior reports show the efficacy of curcumin in improving skin aging and disorders such as acne, psoriasis, infection, skin inflammation, and skin cancer due to its antioxidative, antibacterial, and anti-inflammatory properties. Research of curcumin has shown it to be useful in treating photodamage, skin cancer, wound healing, psoriasis, inflammation, and acne.

While curcumin is commonly used as an antioxidant and an anti-inflammatory compound both topically and orally, its chemical instability, water insolubility, and limited tissue distribution limits its bioactivity significantly. Scientists have tried to use various technologies to solve its insolubility and instability issues. However, not much research has been done on the improvement of its photostability as a cosmetic ingredient, and there is limited information on whether curcumin will be a stable ingredient in consumer products.

### SUMMARY OF THE INVENTION

In one embodiment, the present invention addresses that need with a composition comprising a complex of curcumin with a polyphenolic macrocyclic host, wherein the macrocyclic host is a resorcin[4]arene. In another embodiment, the polyphenolic macrocyclic host is calix[8]arene. In one embodiment, the polyphenolic macrocyclic host is tert-butylcalix[8]arene. In another embodiment, the polyphenolic macrocyclic host is tert-butylcalix[4]arene. In one embodiment, the polyphenolic macrocyclic host is p-PO3-calix[8]arene.

In one embodiment, the present invention is a skincare formulation comprising a complex of curcumin with a polyphenolic macrocyclic host, wherein the macrocyclic host is a resorcin[4]arene. In another embodiment, the polyphenolic macrocyclic host is calix[8]arene. In one embodiment, the polyphenolic macrocyclic host is tert-butylcalix[8]arene. In another embodiment, the polyphenolic macrocyclic host is tert-butylcalix[4]arene. In one embodiment, the polyphenolic macrocyclic host is p-PO3-calix[8] arene.

In another embodiment, the complex comprises from about 4 to about 8% by weight of the formulation. In one embodiment, the complex comprises from about 2 to about 10% by weight of the formulation

In another embodiment, skincare formulation also includes a cosmetically acceptable carrier. In one embodiment, the carrier comprises one or more carriers selected from the group consisting of preservatives, emollients, emulsifying agents, surfactants, moisturizers, gelling agents, thickening agents, conditioning agents, film-forming agents, stabilizing agents, anti-oxidants, texturizing agents, gloss agents, mattifying agents, solubilizers, pigments, dyes, and fragrances.

In another embodiment, the present invention is a method of treating a subject to prevent, treat or reverse acne or post-acne lesions. The method involves applying a therapeutically effective amount of a composition comprising a complex of curcumin with a polyphenolic macrocyclic host, wherein the macrocyclic host is a resorcin[4]arene, to the skin of a subject in need of such treatment.

In one embodiment, the present invention is a method of lightening the skin of a subject. The method involves applying a therapeutically effective amount of a composition comprising a complex of curcumin with a polyphenolic macrocyclic host, wherein the macrocyclic host is a resorcin[4]arene, to the skin of a subject in need of such treatment

In another embodiment, the present invention is a method of preparing a complex of curcumin and calix[8]arene. The method involves combining curcumin with calix[8]arene in a Vortex Fluidic Device.

### BRIEF DESCRIPTION OF THE DRAWINGS

The foregoing summary, as well as the following detailed description of preferred embodiments of the application, will be better understood when read in conjunction with the appended drawings.

FIG. 1A is an image showing the molecular structure of curcumin.

FIG. 1B is an image showing the molecular structure of TBC₈.

FIG. 2A is a graph showing full NMR titration spectra of curcumin+TBC₈ at different mol ratios in CDCl₃.

FIG. 2B is a graph showing zoomed in spectra to show the left shifts of —OH.

FIG. 2C is a graph showing zoomed in spectra to show the right shifts of 6, 6′.

FIG. 2D is a graph showing zoomed in spectra to show the right shifts of —OCH3.

FIG. 3 is an illustration showing the possible confirmation of the complex of curcumin+TBC₈.

FIG. 4 is a graph showing the NMR spectrum of curcumin, calix[8]arene and curcumin+calix[8]arene in CDCl₃.

FIG. 5 is a graph showing the NMR spectrum of curcumin, p-PO₃-calix[8]arene and curcumin+p-PO₃-calix[8]arene in CDCl₃.

FIG. 6 is a graph showing the NMR spectrum of curcumin, TBC₄ and curcumin+TBC₄ in CDCl₃.

### DETAILED DESCRIPTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The term “therapeutically effective amount” means an amount of a compound according to the disclosure which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the disclosure which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the disclosure, and the age, body weight, general health, sex, and diet of the patient. Such a therapeutically effective amount can be determined routinely by one of ordinary skill in the art having regard to their own knowledge, the prior art, and this disclosure.

The present invention relates to the complexation of a supramolecular host and guest cocrystal which contains a polyphenolic macrocycle resorcin[4]arene as host and an anti-inflammatory, anti-bacterial, anti-oxidant and skin lightening agent, curcumin, for a variety of skin and medical applications. In various embodiments, the polyphenolic macrocycles are tert-butylcalix[8]arene, tart-butylcalix[4]arene, calix[8]arene or p-PO3-calix[8]arene. In another embodiment, curcumin complexation with calix[8]arene is achieved in a novel way through a shear thinning device called VFD (Vortex Fluidic Device). These complexes can have implications in skin acne, skin lightening, and in health related applications.

In one embodiment, the present invention is a complex of curcumin with a polyphenolic macrocyclic host through non-covalent supramolecular interactions for skin and health related applications. The present invention of host and guest is moderately lipophilic in nature based on molecular structure. The lipophilic nature helps in skin permeation for localized effect as required in treating acne topically. Chemically, they are linked by a non-covalent bond which helps in dissociation and provide individual biological effects such as antioxidant, anti-inflammatory, anti-bacterial and anti-scarring.

### Curcumin

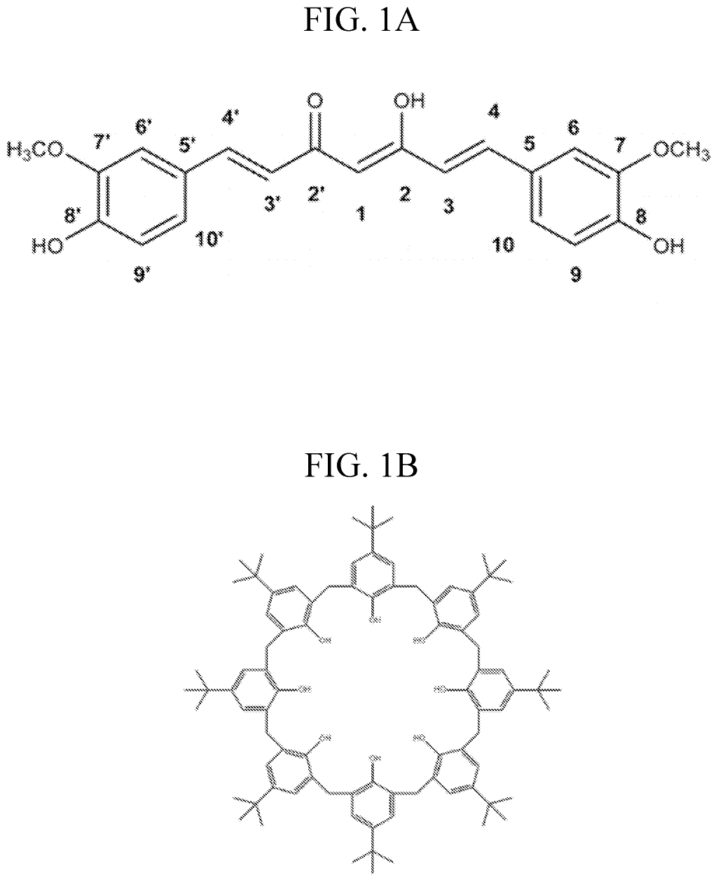

The complexes of the present invention incorporate curcumin. Curcumin (diferuloylmethane, FIG. 1A) is an antioxidant polyphenol from the plant Curcuma longa and is commonly used as a spice component. It has a number of useful properties, including utility as an anti-inflammatory, an anti-bacterial, an anti-oxidant and as a skin lightening agent and/or acne treatment.

Curcumin's anti-inflammatory properties have been established in prior studies. The primary mechanism by which curcumin modulates inflammation is by reducing the expression of the two main cytokines that are released by monocytes and macrophages. These molecules are interleukin 1 (IL-1) and TNF-α, which have important roles in the regulation of the inflammatory response. Also, curcumin inhibits the activity of the proinflammatory transcriptional factor, NF-κB, which is responsible for the regulation of many genes involved during the initial onset of the inflammatory response. A variety of kinases (AKT, PI3K, and IKK) activate NF-κB. Suppression of NFκB activation causes downregulation of cyclooxygenase-2 and inducible nitric oxide synthase, and prevents upregulation of vascular endothelial growth factor (VEGF) messenger RNA and microvascular angiogenesis during inflammatory conditions. Curcumin's anti-inflammatory actions can be utilized to control inflammation of the skin resulting from different skin diseases.

Curcumin also has a broad spectrum of antimicrobial activities against different bacteria, viruses, fungi, and parasites. It has been shown to kill several pathogenic gram-positive bacteria such as Staphylococcus aureus, Staphylococcus epidermidis and Enterococcus that cause infections such as skin diseases, pneumonia, meningitis and urinary tract infections in human beings. It has been suggested that curcumin inhibits bacterial cell division.

Exposure of human skin to solar radiation, chemical pollutants, and mechanical stress results in the generation of free radicals. Free radicals, like reactive oxygen species (ROS), are unstable chemical entities that are highly reactive, cause skin damage through inflammation, and may result in skin cancer. The resultant destruction of proteins, collagen, and elastic fibers is reflected in the signs of skin aging. Antioxidants are compounds that are protective by quenching free radical activity. The antioxidant system in the skin includes superoxide dismutases (SOD), catalases, and peroxidases (selenium-dependent glutathione peroxidases, for example), Aging and prolonged exposure to ROS-generating factors, which include poor nutrition, alcohol intake, UV radiation, stress, and environmental pollution, result in ROS accumulation, which in turn damages the skin.

While most of the antioxidants have either a phenolic functional group or a-diketone group, there are different functional groups including the B-diketo group, carbon-carbon double bonds, and phenyl rings containing varying amounts of hydroxyl and methoxy entities that make curcumin a unique and potent antioxidant. Curcumin's antioxidant activity is attributed to its diketone and phenol moieties (diferuloyl-methane portion of the molecule), which are free radical quenchers. It has been proposed that the antioxidant mechanism of curcumin includes an oxidative coupling reaction at the 3′ position of the curcumin structure with lipid and a subsequent intramolecular Diels-Alder reaction. Curcumin functions as a mediator in the regulation of genes related to the generation of proteins with antioxidant characteristics such as here oxygenase-1, transcription and growth factors, and inflammatory cytokines. Curcumin also regulates antioxidant enzymes, scavenges hyperglycemia-induced ROS, and profoundly increases the intracellular antioxidant, reduced glutathione (GSH), which serves to decrease lipid peroxidation. Studies have also shown strong protective effects of curcumin against damage to the keratinocytes and fibroblasts in the skin induced by 1-1202. Tetrahydrocurcuminoids obtained by hydrogenating curcuminoids are one of the major colorless metabolites of curcumin, in the form of its glucuronide conjugate in bile. This conjugate compound has been shown to have enhanced antioxidant properties with superior free radical scavenging, free radical formation prevention, and increased lipid peroxidation inhibition compared to curcumin and vitamin E, This suggests that curcumin and its derivatives may be promising and effective antioxidants.

Since skin hyperpigmentation is a major concern in skin aesthetics, skin lightening agents have been used widely, especially in China. Accumulating evidence suggests that curcumin has a diverse range of molecular targets, some of which are involved in melanogenesis-regulating pathways. It was recently reported that curcumin suppressed melanogenesis in B16 melanoma cells. This shows the inhibitory effects of curcumin on melanogenesis in normal human melanocytes.

Further, curcumin's anti-inflammatory and antimicrobial properties make it an ideal candidate for acne treatment. Prior studies have found it to be effective at inhibiting S. epidermidis (a bacteria involved in acne). Thus, curcumin is a promising therapeutic agent for the topical treatment of acne vulgaris. Also, in other studies, an emulsion containing curcumin showed antibacterial activity against propionibacteria species (the primary agent involved in inflammatory acne).

### Supramolecular Hosts

In various embodiments of the present invention, the supramolecular host of the inventive complex is a polyphenolic macrocycle resorcin[4]arene. Useful polyphenolic macrocycles include calix[8]arene, p-tert-butylcalix[4]arene, p-tert-butylcalix[8]arene and p-PO3-calix[8]arene.

The field of supramolecular chemistry has engaged in relentless development for several years, in which macrocyclic cavitands are crucial units. Familiar illustrations of such synthetic supramolecular cavitands incorporate cyclodextrins, calixarenes and analogs, pillarenes, and cucurbiturils. These cavitands basically contrast in symmetry, shape, and hydrophilicity. Among other macrocycles, the family of macrocycles known as calixarenes has held particular importance due to their bowl-shaped conformation. They are considered to be the model for host-guest binding and self-assembly. Calixarenes are phenol-based macrocycles, whereas their sister derivatives (resorcinarenes and pyrogalloarene) are synthetic polyphenols. The calixarene molecules may be characterized as calix(N)arenes in which N is an integer within the range of 4-8. Thus the calixarenes ranging from calix(4)arene to calix(8)arene and their derivatives can be employed in carrying out the invention. The calixarene molecules may be distally substituted with a substituent selected from the group consisting of methyl, ethyl, propyl, butyl amyl or phenyl groups.

p-tert-Butylcalix[4]arene is a bowl-shaped molecule capable of forming a range of polynuclear metal clusters under different experimental conditions. p-tert-Butylcalix[8]arene (TBC₈) is a significantly more flexible analogue to p-tert-Butylcalix[4]arene (TBC₄).

### Formulations

The complexes of the present invention can be included in a number of formulations, including formulations for the treatment of acne, skin lightening treatments, antibacterial formulations, anti-inflammatory formulations, and anti-oxidant formulations.

In an embodiment, a formulation for use according to the invention is suitable for topical or local application to the skin, in particular human skin. The ingredients are combined with a “cosmetically-acceptable topical carrier,” i.e., a carrier for topical use that is capable of having the other ingredients dispersed or dissolved therein, and possessing acceptable properties rendering it safe to use topically. A formulation which is “suitable for” topical or local application may also be adapted for topical or local application.

The carrier is that material or combination of materials that is used to essentially carry or deliver the complexes of the present invention to the skin. The specific carrier material will depend upon the delivery method itself. A formulation for use according to the invention may be in the form of a fluid, for example a lotion, cream, ointment, varnish, foam, paste, gel or other viscous or semi-viscous fluid, or a less viscous fluid such as might be used in sprays or aerosols. It may take the form of a solution, suspension or emulsion. It may take the form of a powder or of granules, which may be designed to be added to liquid (e.g. water) prior to use. Each composition will typically include any of the known topical excipients and like agents necessary for achieving the particular form; though it will be recognized that the components of such carriers will be or should be dermatologically-acceptable materials. Suitable excipients include, e.g., mineral oils and emulsifying agents. Simple embodiments include carriers of water, alcohol or water/alcohol combinations, or other solvent(s) or solvent systems in which the aforementioned actives may be, e.g., soluble, dispersed, emulsified, etc. In other embodiments, the formulations include excipients and the like that create substantially stable, homogenous compositions and/or provide body and viscosity to the formulations so that the actives do not merely run off the skin once applied. In some embodiments, the carrier will comprise from about 30 to about 99% by weight of the formulation. Generally speaking, any known carrier or base composition employed in traditional cosmetic and/or dermatological applications/compositions may be used in the practice of the present invention. Those skilled in the art will readily recognize and appreciate what carriers may be employed in light of the intended form and/or delivery method for the inventive formulations.

In an embodiment the formulation is, or may be, applied to a substrate such as a sponge, swab, brush, pad, tissue, cloth, wipe, skin patch or dressing (which includes a bandage, plaster, skin adhesive or other material designed for application to a tissue surface), to facilitate its administration.

The compositions of the present invention may be prepared using methodology that is well known by an artisan of ordinary skill in the field of cosmetics formulation. In one embodiment, the method comprises the simple mixing of the components; though, especially where insoluble or immiscible components are employed higher agitation or homogenization may be necessary to prepare an appropriate composition, e.g., an emulsion or suspension, etc. Additionally, during the preparation, it may be desirable to add known pH adjusters to in order to maintain a proper pH of the composition for topical application, especially if basic ingredients are to be employed. Generally, the pH should be on the neutral to slightly acidic side, perhaps as low as pH 4. Preferably, though, the pH will be in the range of from about 5 to about 6.5.

The complexes of the present invention may be included in formulations at various concentrations, depending on the specific end use and carrier. In one embodiment, the complexes comprise from about 2 to about 10 wt % of a formulation. In another embodiment, the complexes comprise from about 4 to about 8 wt % of a formulation.

### Skin Lightening

In one embodiment, the complexes of the present invention are used as actives in a skin lightening formulation. In some embodiments, the skin lightening compositions of the present invention will also contain a second skin lightening/even-toning ingredient other than the supramolecular host-curcumin complexes. A non-limiting list of suitable examples includes coconut water, coconut milk, palm water, palm nut milk, pecan nut milk, almond nut milk, cashew nut milk, walnut nut milk, and concentrates of the foregoing, or any combinations of the foregoing. Other non-limiting skin lightening ingredients are, for example, Phyllanthus emblica fruit extract, bearberry extract, Mulberry extract, Licorice extract, Propolis extract, aceroal cherry fermentate, cucumber extract, Green tea poly phenols, Grape seed extract, Pine bark polyphenols, resveratrol, oxyresveratrol, stilbenes, coumarins, flavonoids, niacin amide, anthraquinones, xanthones, lignans, glabridin, curcurmine, dihydrocucurmine, epigallocatechin-3-gallate, hydroxyl benzoic acids or their derivatives, tomato glycolipids, perilla plant, Ligusticum lucidum extract, sugar amines and combinations thereof.

In other embodiments, the skin lightening formulations of the present invention also include sugar amines, which are also known as amino sugars and are to be employed in a safe and effective amount. Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt). Examples of sugar amines that are useful herein include glucosamine, N-acetyl glucosamine, glucosamine sulfate, mannosamine, N-acetyl mannosamine, galactosamine, N-acetyl galactosamine, their isomers (e.g., stereoisomers), and their salts (e.g., HCl salt). Preferred ingredients are glucosamine, particularly D-glucosamine and N-acetyl glucosamine, particularly N-acetyl-D-glucosamine. Yet another group of skin lightening agents are the N-acyl amino acid compounds, including, but are not limited to, N-acyl phenylalanine, N-acyl tyrosine, their isomers, including their D and L isomers, salts, derivatives, and mixtures thereof. The skin lightening agents of this paragraph may be used alone or in combination with other skin lightening agents mentioned above.

The second skin lightening agent will be present in a safe and effective amount, generally an amount sufficient to induce the desired effect of lightening. The specific amount will vary depending upon the type of agent and the nature and level of desired effect. In one embodiment, the lightening agents are present in an amount of from about 0.01 wt % to about 20 wt %.

### EXAMPLES

### Example 1: Curcumin+TBC₈ Complex NMR Titration

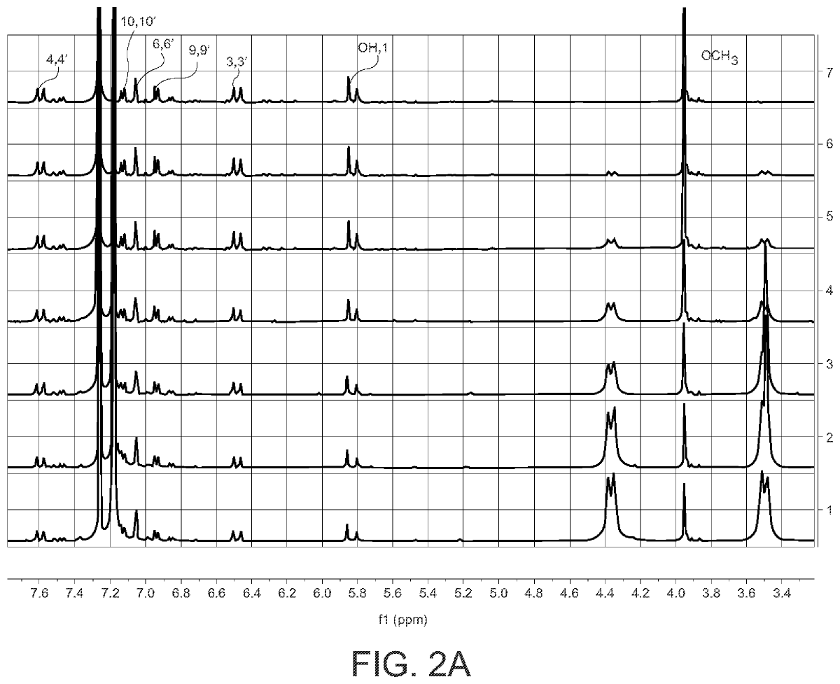

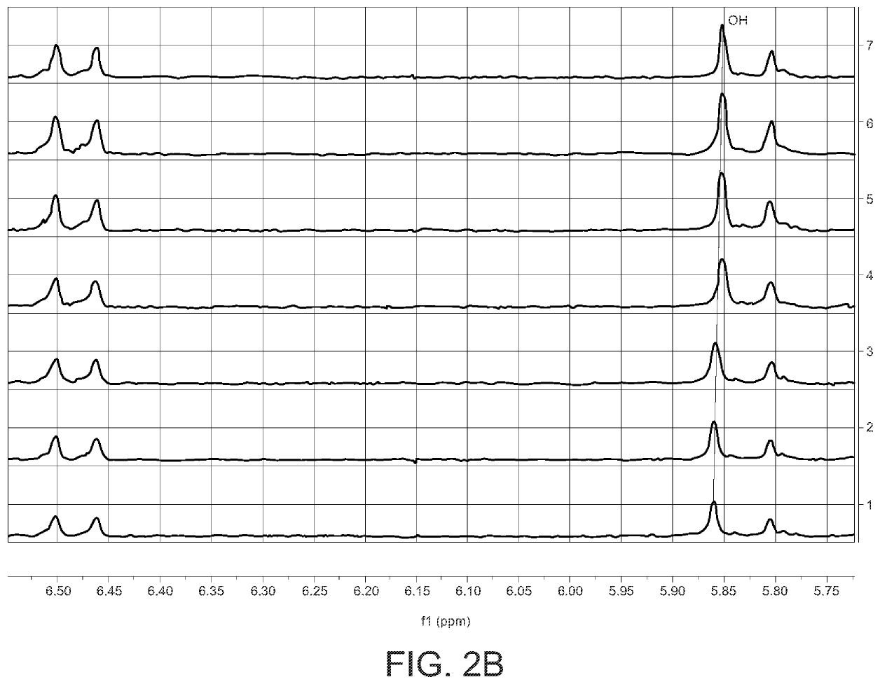

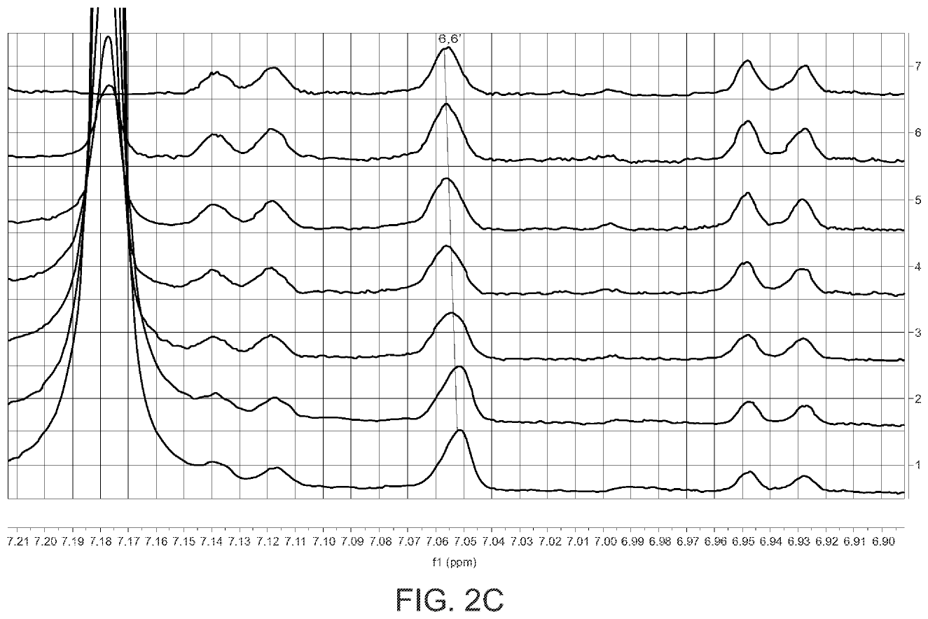

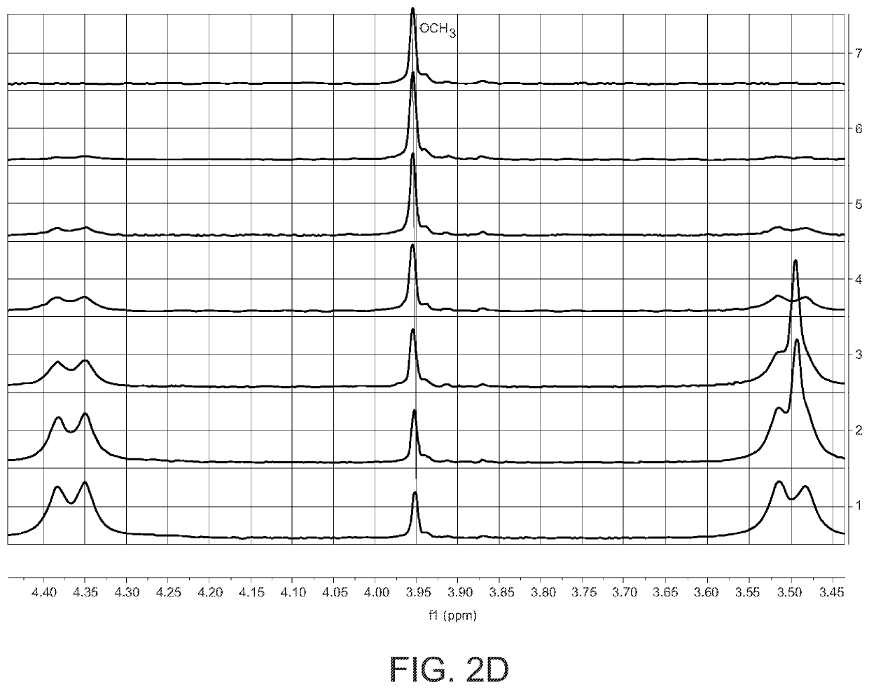

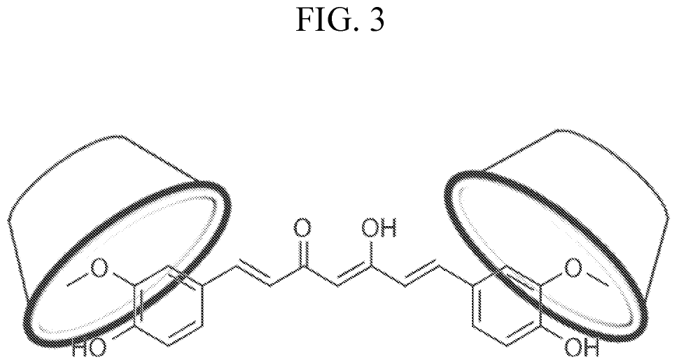

Curcumin and TBC₈ were mixed in CDCl₃. The NMR titration (FIGS. 2A-D) showed changes in curcumin aromatic protons (6 and 6′). Aromatic protons 6, 6′ and protons on —OCH3 showed a shielding effect in presence of TBC₈. On the other hand, —OH showed a left shift in presence of TBC₈. These observations indicate the inclusion of curcumin in the TBC₈ cavity. FIG. 3 predicts the possible confirmation of the complex.

Instead of curcumin+TBC₈, there are several other complexes made with macrocycles with similar structures described in the examples below, which shifts were also found.

### Example 2: Curcumin+Calix[8]arene in CDCl₃ (VFD)

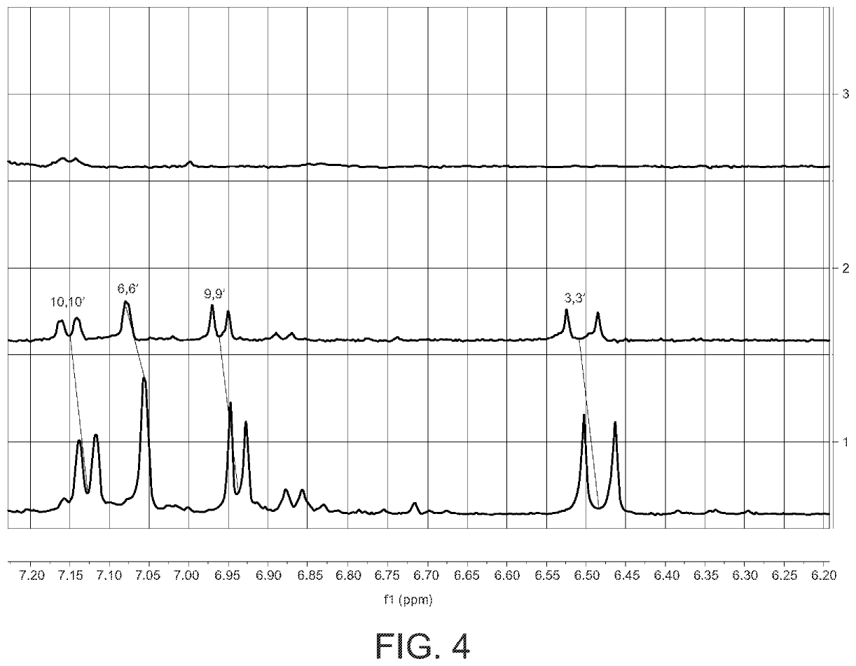

Curcumin and calix[8]arene were mixed in CDCl₃. Because of the low solubility of calix[8]arene, VFD was used to create a thin layer of compounds in the solvent and increase the solubility. The NMR spectra (FIG. 4) showed changes in curcumin aromatic protons (10, 10′, 6, 6′, and 9, 9′). Aromatic protons 10, 10′, 6, 6′, and 9, 9′ and protons on 3, 3′ in the presence of calix[8]arene. These observations indicate the inclusion of curcumin in the calix[8]arene cavity.

### Example 3: Curcumin+p-PO₃-calix[8]arene in CDCl₃

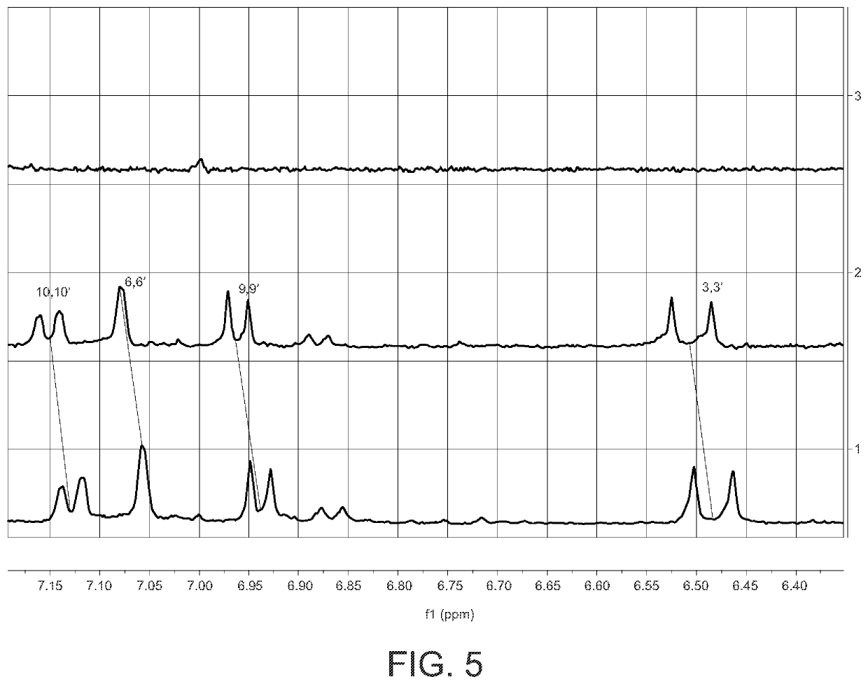

Curcumin and p-PO₃-calix[8]arene were mixed in CDCl₃. Similarly, the NMR spectra (FIG. 5) showed changes in curcumin aromatic protons (10, 10′, 6, 6′, and 9, 9′). Aromatic protons 10, 10′, 6, 6′, and 9, 9′ and protons on 3, 3′ showed a shielding effect in presence of p-PO3-calix[8]arene. These observations indicate the inclusion of curcumin in the p-PO₃-calix[8]arene cavity.

### Example 4: Curcumin+TBC₄ in CDCl₃

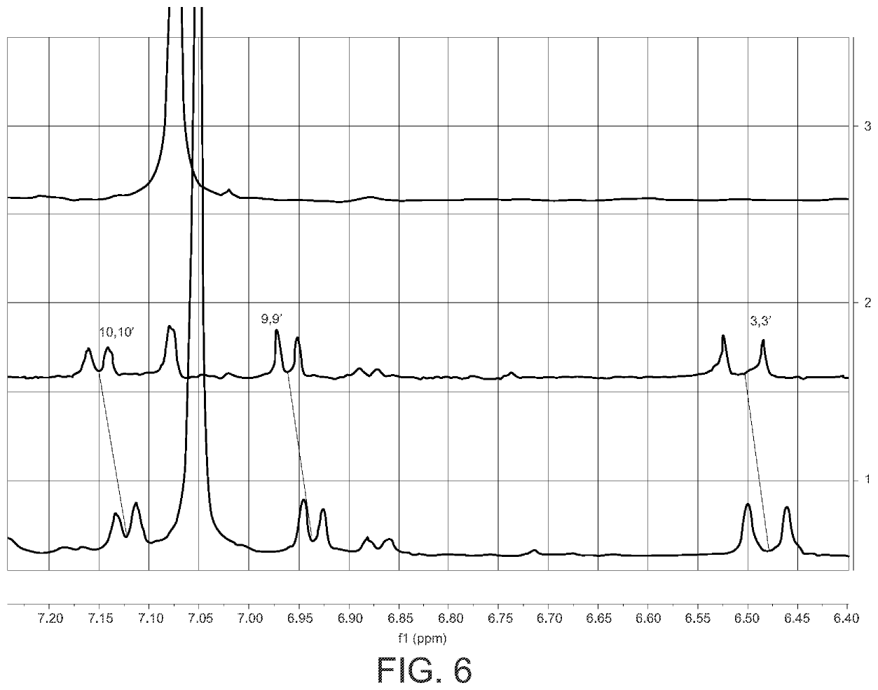

Curcumin and TBC₄ were mixed in CDCl₃. Similarly, the NMR spectra (FIG. 6) showed changes in curcumin aromatic protons (9, 9′ and 10, 10′). Aromatic protons 9, 9′, 10, 10′ and protons on 3, 3′ showed a shielding effect in presence of TBC₄. These observations indicate the inclusion of curcumin in the TBC₄ cavity.

After the initial screening, it was found that curcumin could make complex with TBC₈, calix[8]arene, p-PO₃-calix[8]arene, and TBC₄. Several organic solvents were tried and CDCl₃ was found the most suitable. Although still had a low solubility, TBC₈ was the most soluble in organic solvents, so the titration was carried out using the curcumin+TBC₈ complex. The complex with calix[8]arene, p-PO₃-calix[8]arene, and TBC₄ showed similar results which indicated that those three macrocycles impacted curcumin as a guest in similar ways.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A composition comprising a complex of curcumin with a polyphenolic macrocyclic host, wherein the macrocyclic host is a resorcin[4]arene.

2. The composition of claim 1 wherein the polyphenolic macrocyclic host is calix[8]arene.

3. The composition of claim 1 wherein the polyphenolic macrocyclic host is test-butylcalix[8]arene.

4. The composition of claim 1 wherein the polyphenolic macrocyclic host is tert-butylcalix[4]arene.

5. The composition of claim 1 wherein the polyphenolic macrocyclic host is p-PO3-calix[8]arene.

6. A skincare formulation comprising a complex of curcumin with a polyphenolic macrocyclic host, wherein the macrocyclic host is a resorcin[4]arene.

7. The skincare formulation of claim 6 wherein the polyphenolic macrocyclic host is calix[8]arene.

8. The skincare formulation of claim 6 wherein the polyphenolic macrocyclic host is tert-butylcalix[8]arene.

9. The skincare formulation of claim 6 wherein the polyphenolic macrocyclic host is tert-butylcalix[4]arene.

10. The skincare formulation of claim 6 wherein the polyphenolic macrocyclic host is p-PO3-calix[8]arene.

11. The skincare formulation of claim 6, wherein the complex comprises from about 4 to about 8% by weight of the formulation.

12. The skincare formulation of claim 6, wherein the complex comprises from about 2 to about 10% by weight of the formulation.

13. The skincare formulation of claim 6, further comprising a cosmetically acceptable carrier.

14. The skincare formulation of claim 13, wherein said carrier comprises one or more carriers selected from the group consisting of preservatives, emollients, emulsifying agents, surfactants, moisturizers, gelling agents, thickening agents, conditioning agents, film-forming agents, stabilizing agents, anti-oxidants, texturizing agents, gloss agents, mattifying agents, solubilizers, pigments, dyes, and fragrances.

15. A method of treating a subject to prevent, treat or reverse acne or post-acne lesions comprising applying a therapeutically effective amount of the composition of claim 1 to the skin of a subject in need of such treatment.

16. A method of lightening the skin of a subject comprising applying a therapeutically effective amount of the composition of claim 1 to the skin of a subject in need of such treatment.

17. A method of preparing a complex of curcumin and calix[8]arene comprising combining curcumin with calix[8]arene in a Vortex Fluidic Device.
